(19) United States
(12) Patent Application Publication
(10) Pub. No.: US 2026/0020843 A1
(43) Pub. Date: Jan. 22, 2026

(54) TREATMENT OF MALE REPRODUCTIVE DISORDER

(71) Applicant: Imperial College Innovations Ltd, London (GB)

(72) Inventors: Channa JAYASENA, London (GB); Mengxing TANG, London (GB); Adrian LIM, London (GB); Suks MINHAS, London (GB); Jipeng YAN, London (GB)

(73) Assignee: Imperial College Innovations Ltd, London (GB)

(21) Appl. No.: 19/289,894

(22) Filed: Aug. 4, 2025

Related U.S. Application Data
(63) Continuation-in-part of application No. PCT/GB2024/050292, filed on Feb. 2, 2024.

(30) Foreign Application Priority Data
Feb. 3, 2023 (GB) 2301589.4

(51) Int. Cl. A61B 8/00; A61B 8/08

(52) U.S. Cl. CPC A61B 8/5223; A61B 8/0891; A61B 8/469; A61B 8/481; A61B 8/5276

(57) ABSTRACT

The present invention relates to the use of ultrasound imaging techniques and computer implemented image processing methods for determining and assessing male infertility, as well as non-invasive diagnostic methods to identify and diagnose male infertility, such as hypogonadism (testosterone deficiency). Furthermore, the invention relates to methods of selecting male subjects for fertility treatment and methods for the treatment of these subjects.

### CROSS-REFERENCE TO RELATED APPLICATIONS

This application is a continuation-in-part of International Application No. PCT/GB2024/050292, filed Feb. 2, 2024, which in turn claims the priority benefit of United Kingdom Application No. 2301589.4, filed Feb. 3, 2023. Each of these applications is incorporated herein by reference in its entirety for all purposes.

### FIELD OF THE INVENTION

The present invention relates to the field of medicine, in particular to the field of male infertility or sub-optimal fertility and the treatment thereof. Specifically, the instant application relates to methods for diagnosing or aiding in the diagnosis of hypogonadism and methods for selecting a male subject for fertility treatment using novel ultrasound scanning techniques to investigate microvessel biomarkers.

### BACKGROUND

Around 15% of the global population is affected by infertility, wherein male infertility is known to contribute to 20-70% of all the cases. It has further been estimated that 2.5-12% of all men are infertile (Agarwal et al., 2015). Male infertility is the reduction of sperm number, function, or quality in semen, which reduces the chances of a couple being able to have a baby after 12 months of regular unprotected intercourse (Wall and Jayasena, 2018). The inventors have developed a method of identifying those men with future fertility problems by testing the function of the testes. Without such as test, they would remain asymptomatic and be unaware of their poor semen quality, which could deteriorate. Identified men could undergo sperm freezing to preserve their fertility, or their sperm could be used during in vitro fertilisation or intracytoplasmic injection with the eggs from their female partner, to treat a fertility problem.

Male Hypogonadism is a clinical syndrome that results from a variety of patho-physiological conditions in which testosterone concentration is diminished below the normal range and/or there is a decrease in sensitivity to testosterone (Bhasin et al., 2018). Specifically, male Hypogonadism may involve problems associated with either the testes or with the signal from the brain that controls testosterone secretion. Testosterone is the major circulating androgen in men. More than 95% of the 6-7 mg of testosterone produced per day is secreted by the approximately 500 million Leydig cells in the testes. Two hormones produced by the pituitary gland, luteinizing hormone (“LH”) and follicle stimulating hormone (“FSH”), are required for the development and maintenance of testicular function.

Today, male hypogonadism is the most common hormone deficiency in men, affecting 5 in every 1,000 men. For example, male Hypogonadism is one of the main causes of male infertility and generally tends to have a higher prevalence in older men, obese men, and men with type 2 diabetes. It is estimated that approximately 35% of men older than 45 years of age and 30-50% of men with obesity or type 2 diabetes have hypogonadism.

Hypogonadism can generally be classified into three types. Primary hypogonadism includes the testicular failure due to congenital or acquired anorchia, XYY Syndrome, XX males, Noonan's Syndrome, gonadal dysgenesis, Leydig cell tumours, maldescended testes, varicocele, Sertoli-Cell-Only Syndrome, cryptorchidism, bilateral torsion, vanishing testis syndrome, orchiectomy, Klinefelter's Syndrome, chemotherapy, toxic damage from alcohol or heavy metals, and general disease (renal failure, liver cirrhosis, diabetes, myotonia dystrophy). Secondary hypogonadism involves an idiopathic gonadotropin or LH-releasing hormone deficiency. This type of hypogonadism includes Kallman's Syndrome, Prader-Labhart-Willi's Syndrome, Laurence-Moon-Biedl's Syndrome, pituitary insufficiency/adenomas, Pasqualini's Syndrome, hemochromatosis, hyperprolactinemia, or pituitary-hypothalamic injury from tumours, trauma, radiation, or obesity. These men have low testosterone serum levels but have gonadotropins in the normal to low range. Tertiary hypogonadism may be age-related. Men experience a slow but continuous decline in average serum testosterone after approximately age 20 to 30 years. Tertiary or late-onset hypogonadism (LOH) has been defined as a complex cluster of symptoms seen in aging men accompanied by a decrease in serum testosterone levels. Cross-sectional studies in men have found that the mean testosterone value at age 80 years is approximately 75% of that at age 30 years. Despite these definitions, identification of these men is complicated and difficult.

Other risk factors that may lead to male hypogonadism include genetic anomalies or the use of alkylating agents (chemotherapeutic drugs), opioids or glucocorticoids (GC). As shown by Antony et al. (2020), opioid-induced hypogonadism (OHG) resulted upon long-term opioid therapy and patients with OHG were presented mainly by sexual dysfunction and infertility. Likewise, GC exposure (e.g., dexamethasone) significantly decreased the total and free testosterone levels (Mohammed et al., 2020). Key diagnostic symptoms aside from low testosterone include, e.g., erectile dysfunction, decreased libido, and gynaecomastia. Other diagnostic factors include decreased energy and fatigue, delayed puberty, lack of scrotal hyper-pigmentation and rugae, or decreased muscle mass and strength. Over time, and if remained untreated, male hypogonadism may lead to reduced muscle growth, reduced male pattern body hair, reduced bone density (e.g., osteoporosis) and anaemia.

At present, several clinical signs in conjunction with laboratory test values, i.e., blood test, are considered when trying to determine whether a patient is testosterone deficient and/or to diagnose male hypogonadism. In particular, an ‘early morning serum total testosterone level’ of less than 10.4 nanomol/L (<300 nanograms/dL) on at least two separate occasions in a symptomatic man generally confers the diagnosis of hypogonadism (used as the “gold-standard” test to diagnose male hypogonadism). Other diagnostic factors that are considered include serum sex hormone binding globulin (SHBG), serum free testosterone, serum bioavailable testosterone, and serum gonadotrophins luteinizing hormone (LH) and follicle-stimulating hormone (FSH). In other cases, karyotypes have been employed to diagnose chromosomal abnormalities, such as Klinefelter's syndrome.

Despite these different approaches, diagnosis of male hypogonadism has remained controversial. This is not only because many of the symptoms of male hypogonadism are non-specific but also because the clinical signs and symptoms of hypogonadism vary depending on whether the patient presents before or after puberty. Moreover, the current ‘gold-standard’ test lacks a universally accepted threshold for determining whether a man has hypogonadism (Jayasena et al., 2022). Consequently, some men are deprived of appropriate testosterone treatment, and other men are inappropriately treated with testosterone, which may increase the risks of other disorders such as, e.g., erythrocytosis (high red cell count), blood clot risk and/or oedema. Hence, there is a need to develop improved methods to diagnose men with hypogonadism and to select these men for fertility treatment.

Angiopathy is the generic term for a disease of the blood vessels and can further be categorized in macroangiopathy and microangiopathy. Male reproductive organs associated with pathologies such as hypogonadism may be associated with abnormalities in blood vessels' characteristics. For example, varicocele (dilated veins of the pampiniform plexus) is a relatively common problem in patients who seek medical attention for infertility problems and refers to a swelling of the veins that drain the testicle. It's the most common reversible cause of male infertility. Although the exact reason that varicoceles cause infertility is unknown, it may be related to abnormal blood flow. Varicoceles lead to reduced sperm quantity and quality (Bertolotto et al., 2020).

The use of ultrasonography or ultrasound (US) has become an important component in the evaluation and treatment of male reproductive tract disorders. For instance, ultrasound (US) is the imaging modality of choice for varicocele evaluation. Methods and systems relating to the detection of viable sperm in subjects having at least one testicle using high frequency ultrasound imaging are described in patent application WO2008/052328A1. Schurich et al. (2009) measured testicular volume using B-mode US and showed that testicular volume correlated significantly with testicular function. That is, follicle-stimulating hormone (FSH) was inversely correlated with testicular volume and directly correlated with testicular vascularization, suggesting that ultrasonographic and colour Doppler scanning of the testes may be used, if a sperm count is not available, to indirectly assess the gonadal function. Foresta et al. (1998) have shown that colour Doppler sonography of the testis may be useful in the differential diagnosis of azoospermia.

It is known from the art that microcirculation has a significant role in disease development and progression. Microvessels are crucial for supplying oxygen and nutrients to tissues, making the imaging of microvessels an ideal method of working out how well any given tissue of the body is working. In case of hypogonadism, the reproductive organs are associated with abnormalities in microvessel characteristics, which, however, cannot be detected in people using any of the currently available technologies. Therefore, there is presently no method to assess, diagnose or predict, which men have or will develop hypogonadism.

Super-Resolution Ultrasound (SRUS) has become a tool for in vivo microvascular imaging and has been used to study structural vascular alterations in different diseases, including chronic kidney disease, cancer, and atherosclerosis etc. (Andersen et al., 2022). The inventors of the present application have previously described a SRUS method comprising transmitting an ultrasound excitation signal from each element of a transducer array and receiving a response signal from each element of a transducer array (WO2017/216578A1). Systems and methods for super-resolution ultrasound imaging of microvessels in a subject are described in US2020/0178939A1. However, adoption in this space has not been quick since there are good imaging alternatives like MRI imaging.

Contrast-enhanced Ultrasound (CEUS) is an ultrasound examination that uses microsphere or microbubble ultrasound contrast agents (UCA) to better visualize organs and blood vessels. Ultrasound contrast microbubbles are routinely used in the clinic for ultrasound imaging enhancement. Notably, microbubble contrast agents for ultrasound (US) have gained increasing interest in recent years, and contrast-enhanced US (CEUS) is a rapidly evolving field with applications now extending far beyond the initial improvements achieved in Doppler US. This has been achieved as a result of the safe profile and the increased stability of microbubbles persisting in the bloodstream for several minutes (Quaia, 2007).

Microbubbles have a small size (around 2.5 μm), a viscosity similar to blood, and their rheology has been found comparable to that of erythrocytes (Keller et al., 1989). Therefore, MB velocity can be used as a surrogate for blood velocity. As previously reported by Andersen et al. (2022), SRUS imaging can be used to map vessels below 100 μm by tracking microbubbles from arteries/arterioles and separating them from vein/venule tracks using the arterial blood flow direction.

There is a need for improved methods of identifying patients who have hypogonadism or are at risk of low fertility as existing techniques rely on biomarkers that exhibit significant variability or can be subjective.

### SUMMARY OF INVENTION

The present invention generally relates to the use of ultrasound imaging techniques for determining and assessing male infertility, as well as non-invasive diagnostic methods to identify and diagnose male infertility, such as hypogonadism (testosterone deficiency). Furthermore, the invention relates to methods of selecting male subjects for fertility treatment and methods for the treatment of these subjects. The inventors have specifically discovered and developed a new approach for obtaining vascular information that can be used to identify men who would benefit from receiving fertility treatment. Specifically, the inventors conducted an ethics-approved study to investigate the potential of contrast-enhanced ultrasound (CEUS)/super-resolution ultrasound (SRUS) to assess and determine male infertility, i.e., hypogonadism (testosterone deficiency) and—for the first time—provide proof-of-concept evidence that CEUS/SRUS provides an effective method in the detection and assessment of the functional activity of testes. To date, the approach and methods provided herein have not previously been used to assess the microvasculature in reproductive organs, i.e., male testes, and/or identify male subjects that may benefit from fertility treatment as disclosed in the present application. The methods of the invention are all based on the inventors' discovery that CEUS/SRUS can be reliably used in predicting and diagnosing problems of testicular function and to subsequently select male subjects for fertility treatment. Furthermore, the inventors have developed a novel and inventive protocol to measure the function of the testes using microbubble-contrast ultrasound imaging of the microvasculature to predict and diagnose male infertility and low testosterone (hypogonadism).

The inventors have discovered that microbubble ultrasound CEUS/SRUS can be used to assess microvasculature and to assess the function of male reproductive organs. The inventors provide, for the first time, evidence of a proof-of-concept that CEUS/SRUS can be used to detect the function of reproductive organ. Specifically, the inventors provide evidence that CEUS/SRUS can be used for selecting male subjects for fertility treatment and for diagnosing or aiding in the diagnosis of hypogonadism in a male subject. Advantageously, the use of CEUS/SRUS allows the detection of changes in imaging biomarkers such as, e.g., microvessel density (MVD), microvessel tortuosity and microvessel diameter within the testes. Furthermore, the method may be readily automated, using software-implemented image processing methods, to provide an automated method of providing an assessment of testicular function based on input CEUS/SRUS images. The method may also be used to provide an indication of fertility.

Therefore, the present invention provides improved imaging techniques for assessing the microvasculature in reproductive organs in biological males. Specifically, the present invention provides for enhanced imaging of the testicular microvasculature, which has utility in predicting and diagnose problems with testicular function.

The solution to the problem addressed by the instant application of how to provide improved techniques for analysing testicular microvasculature is achieved by the embodiments described herein and defined by the appended claims.

The present invention generally provides methods of selecting a male subject for fertility treatment, methods for diagnosing or aiding in the diagnosis of hypogonadism and methods for assessing testicular function in a male subject. Furthermore, the present invention relates to methods of treatment of male subjects for infertility or sub-optimal fertility, such as the treatment of hypogonadism. The invention also provides methods for analysing testicular microvasculature.

The male reproductive organs, i.e., testes, are ideal for CEUS/SRUS because they are close to a surface that a hand-held ultrasound probe can access. Furthermore, the processing of the CEUS/SRUS images of the testes can be implemented with software methods to provide an at least partially automated method of providing an assessment of testicular function and/or a diagnosis of hypogonadism. Accordingly, in a first aspect there is provided a computer-implemented method for assessing testicular function in a male subject, the method comprising: receiving one or more ultrasound image collected using contrast-enhanced ultrasound (CEUS) and/or super-resolution ultrasound (SRUS); determining one or more testicular parameters from the one or more ultrasound images; comparing the one or more parameters to one or more threshold values of the one or more parameters; determining a difference between the one or more parameters and the one or more threshold values; and outputting an indication of testicular function based on the comparison of the one or more parameters with the one or more threshold values. In further related aspects of the disclosure there are provided a computer program comprising instructions that when executed by the computer, cause the computer to perform the above method and a system comprising a processor configured to perform the computer-implemented method.

Preferably the step of outputting an indication of testicular function based on the comparison of the one or more parameters with the one or more threshold values comprises outputting an indication of fertility or infertility based on the comparison of the one or more parameters with the one or more threshold values. Preferably the step of outputting an indication of testicular function based on the comparison of the one or more parameters with the one or more threshold values comprises outputting a diagnosis of hypogonadism based on the comparison of the one or more parameters with the one or more threshold values. There is often a significant overlap with subjects that have hypogonadism and infertility. The inventors have demonstrated that the determined testicular parameters can be used as a biomarker for one or both of hypogonadism and male infertility.

Preferably the computer-implemented method further comprises determining a region of interest within the one or more ultrasound images and performing image-processing within the region of interest to determine the one or more testicular parameters. The method may comprise receiving a user-input selection of the region of interest, for example within a user interface displaying a received ultrasound image. In other examples the region of interest may be determined automatically, for example by performing feature recognition, for example to determine a region comprising microvessel to be measured.

Preferably the method further comprises outputting a diagnosis of hypogonadism based on based on the comparison of the one or more parameters with the one or more threshold values. In this way the invention provides an automated hypogonadism diagnosis based on the input ultrasound images. In some examples the output may a binary yes/no indication representing whether the subject is more likely or not to have hypogonadism based on the analysis of the extracted testicular parameters. In some examples, the method may comprise outputting an indication of likelihood of the diagnosis, based on the comparison of the parameters to the threshold values, for example based on how close the determined testicular parameters are to the threshold values. In some examples the method may utilise a more complex algorithm, taking as input one or more values of testicular parameter and providing a likelihood of a positive diagnosis based on the input parameters.

Preferably the one or more testicular parameters each comprise a measurement of microvessel morphology based on the one or more ultrasound images. In particular the testicular parameters may each comprise a measurement of an aspect of the microvessel morphology, for example relating to a size, shape, number or density of microvessel. In preferable embodiments the testicular parameters comprise microvessel density, mean microvessel diameter and mean microvessel diameter and the output indication of testicular function is based on said plurality of testicular parameters together.

Preferably the method comprises receiving a sequence of microbubble ultrasound images; processing the sequence of ultrasound images to localise individual microbubbles through a sequence multiple images and thereby determine a plurality of microbubble tracks; determining the measurement of microvessel morphology based on the determined microbubble tracks. The sequence of microbubble images may comprise video data, for example comprises a plurality of frames of the same region of interest. By collecting a sequence of images, microbubbles can be tracked across images and it is possible to resolve microbubbles at a resolution that would otherwise not be possible. This also allows for measurements of the morphology, such as tortuosity to be derived from the microbubble tracks directly.

Preferably the method comprises processing the sequence of ultrasound images to correct for motion in the image by performing image registration across the sequence of images; generating the microvascular image based on the motion-corrected sequence of ultrasound images. Since preferably the images are captured over a period of seconds or minutes it is necessary to correct for inevitable movement of the tissue during the acquisition period to achieve more accurate measurements. This may be achieved using an image registration process.

The one or more testicular parameters are preferably selected from the list comprising: testicular microvessel density, testicular microvessel diameter, testicular microvascular flow, regularity or entropy of testicular microvascular flow, and/or testicular microvessel tortuosity, and spatial heterogeneity of these parameters. The inventors have determined that these parameters are predictive of testicular function, while being readily implemented in an automated images processing procedure.

Preferably the method comprises processing the one or more ultrasound images to determine a microvessel density of the left and/or right testes or the mean thereof; comparing the determined microvessel density to a threshold representing a healthy control; outputting a diagnosis of hypogonadism when the determined microvessel density is reduced compared to the threshold representing a health control.

Preferably the method comprises processing the one or more ultrasound images to determine a mean microvessel density; comparing the determined mean microvessel density to a microvessel density threshold representing a healthy control, wherein the threshold is between 0.05 and 0.15; outputting a diagnosis of hypogonadism when the determined microvessel density is below the microvessel density threshold. The threshold may take another value as specified within the present disclosure. In a preferable embodiment the mean microvessel density threshold value is 0.057

Preferably the method further comprises processing the one or more ultrasound images to determine a microvessel tortuosity of the left and/or right testes or the mean thereof; comparing the determined microvessel tortuosity to a microvessel tortuosity threshold representing a healthy control; outputting a diagnosis of hypogonadism when the determined microvessel tortuosity is greater than the threshold. The microvessel tortuosity threshold may be 3.2 C/L or it may take another value as disclosed in the present disclosure.

The method may further comprise receiving a sequence of microbubble-contrast ultrasound images; processing the sequence of microbubble-contrast images to determine a plurality of microbubble trajectories; calculating the tortuosity of the microbubble trajectories to determine the microvessel tortuosity. By determining the vessel tortuosity directly from the microbubble trajectories rather than from a microvessel image, it is possible to avoid segmentation errors relating to isolating individual vessels for measurement.

Preferably the method further comprises processing the one or more ultrasound images to determine a microvessel diameter in the left and/or right testes or the mean thereof; comparing the determined microvessel diameter to a microvessel diameter threshold representing a healthy control; outputting a diagnosis of hypogonadism when the determined microvessel diameter is reduced compared to the threshold.

Preferably the method comprises processing the one or more ultrasound images to determine a microvessel diameter; comparing the mean microvessel diameter to a mean microvessel diameter threshold, wherein the mean microvessel diameter is between 70 and 120 μm; outputting a diagnosis of hypogonadism when the determined mean microvessel diameter is below the mean microvessel diameter threshold.

Preferably the method comprises processing the one or more ultrasound images to generate a microvessel image; determining the one or more testicular parameters based on the microvascular image. By generating a microvascular image it is possible to measure a wide range of microvascular morphological parameters.

Preferably the method comprises receiving a sequence of ultrasound images collected using contrast-enhanced ultrasound (CEUS) and/or super-resolution ultrasound (SRUS) receiving a sequence of ultrasound images; processing the sequence of ultrasound images to determine a microbubble signal; generating a microvessel image using the determined microbubble signal; calculating the one or more testicular parameters using the microvessel images, wherein the one or more testicular parameters each comprise a measurement of a feature of the microvessel morphology.

In a further aspect, provided herein is a method of selecting a male subject for fertility treatment, wherein the subject has been examined using contrast-enhanced ultrasound (CEUS) and/or super-resolution ultrasound (SRUS), and wherein following examination, one or more testicular parameters have been determined for the subject, wherein the method comprises the following steps in the following order: (a) comparing the one or more parameters of the subject to one or more threshold values of the one or more parameters; (b) determining a difference between the one or more parameters of the subject and the threshold values; and (c) selecting the subject for treatment when the one or more of the parameters of the subject are below or above the threshold values.

Diagnosing hypogonadism can be challenging as this condition presents differently based on your sex at birth and age. Hence, there is an unmet need to develop effective and reliable approaches that allow (early) diagnosis of hypogonadism. The inventors of the present invention have developed a method that allows detection of male infertility (e.g., hypogonadism). The inventors submit that tracking the movement of microbubbles over time can be used to look at microvessels and diagnose male infertility and hypogonadism (testosterone deficiency). Accordingly, in a further aspect provided herein is a method for diagnosing or aiding in the diagnosis of hypogonadism in a male subject, wherein, following examination using contrast-enhanced ultrasound (CEUS) and/or super-resolution ultrasound (SRUS), a test profile of one or more testicular parameters has been determined for the subject; and wherein the method comprises the following steps in the following order: (a) comparing the test profile of the subject to one or more threshold values of the one or more parameters; (b) determining a difference between the test values of the subject and the threshold values; and (c) diagnosing hypogonadism based on the comparison of the test profile with the threshold values.

In a further aspect provided herein is composition for use in treating infertility or sub-optimal fertility of a male subject, wherein the composition comprises an anabolic-androgenic steroid, human chorionic gonadotropin (hCG), clomiphene citrate, aromatase inhibitors, androgen receptor modulators; and wherein the subject has been determined to have reduced density, elevated tortuosity and/or reduced diameter of microvessels within the testes.

In a further aspect provided herein is a method for assessing testicular function in a male subject, the subject having been examined using contrast-enhanced ultrasound (CEUS) and/or super-resolution ultrasound (SRUS), and wherein following examination, one or more testicular parameters have been determined, and wherein the method comprises the following steps in the following order: (a) comparing the one or more parameters to one or more threshold values of the one or more parameters; (b) determining a difference between the one or more parameters and the one or more threshold values; and (c) assessing testicular function based on the comparison of the one or more parameters with the one or more threshold values.

In a further aspect the present invention provides a method of treating male infertility, such as hypogonadism, the method comprising diagnosing infertility using the techniques described herein, and administering suitable treatments, as set out herein.

In a further aspect provided herein is a method of determining the density, tortuosity and/or diameter of microvessels within the testes, using the techniques described herein.

Through the combined use of CEUS and SRUS the inventors submit that it will be possible to effectively assess the function of the testis. CEUS/SRUS will be able to find areas of highest activity in the testes, which are predicted to be most likely to harbour sperm (which could be removed using a biopsy guided by the scan).

Accordingly, in a further aspect provided herein is a method of predicting which men may develop hypogonadism, using the techniques described herein.

### BRIEF DESCRIPTION OF DRAWINGS

FIGS. 1A-1C represent graphs showing microvessel density in male subjects with hypogonadism compared to healthy men (control).

FIGS. 2A-2C represent graphs showing tortuosity of microvessels in male subjects with hypogonadism compared to healthy men (control).

FIGS. 3A-3C represent graphs showing diameter of microvessels in male subjects with hypogonadism compared to healthy men (control).

FIG. 4 is a schematic illustration of a system 10 which includes an ultrasound imaging device 11 and a processor 12.

### DETAILED DESCRIPTION

### Terminology

In order that the present invention may be more readily understood, certain terms are first defined.

Articles “a” and “an” used herein refer to one or to more than one (i.e., to at least one) of the grammatical object of the article. By way of example, “an element” means one element or more than one element, e.g., a plurality of elements.

The terms “about” and “approximately” may be understood to permit standard variation as would be understood by those of ordinary skill in the art.

The term “including” is used herein to mean, and is used interchangeably with, the phrase “including, but not limited to”. Likewise, the term “comprising” is used herein to mean, and is used interchangeably with, the phrase “comprising, but not limited to”.

As used herein, the term “biomarker” or “parameter” refers to a naturally occurring molecule, gene, or characteristic by which a particular pathological or physiological process, disease, etc. can be identified. For example, the biomarker may be selected from multiple parameters including microvessel density, microvascular flow, regularity of the flow, velocity, or tortuosity of the vessels. Tortuosity is expressed as the arc-chord ratio: the ratio of the length of the curve (C) to the distance between its ends (L, length), wherein arc-chord ratio equals 1 for a straight line and is infinite for a circle. The equation should be: t=C/L. These parameters could be utilised as imaging biomarkers alone or in combination. The terms “biomarker” and “parameter” may be used interchangeably. The expression “testosterone or derivative thereof” refers to testosterone, including derivatives such as testosterone ester, testosterone undecanoate, dihydrotestosterone gel.

As used herein, the terms “disease” or “disorder” are used interchangeably to refer to a condition in a subject. In certain embodiments, the condition is a disease in a subject, the severity of which is decreased by inducing an immune response in the subject through the administration of a pharmaceutical composition.

As used herein, the term “effective amount” in the context of administering a therapy to a subject refers to the amount of a therapy which has a prophylactic and/or therapeutic effect(s).

As used herein, the term “in combination” in the context of the administration of two or more therapies, refers to the use of more than one therapy (e.g., more than one prophylactic agent and/or therapeutic agent). The use of the term “in combination” does not restrict the order in which therapies are administered to a subject. Also, as used herein, the term “in combination” may refer to the detection of or the use of one or more than one biomarkers of male infertility together with another biomarker in the diagnosis or assessment of male infertility.

As used herein, the term “pharmaceutically acceptable” means approved by a regulatory agency. The term “carrier” refers to a diluent, adjuvant, excipient, or vehicle with which the pharmaceutical composition is administered.

As used herein, the term “threshold” or “threshold value” refers to a point or level above or below which a result is produced and at which point therapeutic intervention is determined to be needed. It is to be understood that “threshold values” and “threshold” can be used interchangeably. Surpassing or falling below a certain threshold allows differentiation of the test sample from a healthy and/or non-diseased control (e.g., subjects that do not have hypogonadism). The threshold value or threshold can vary depending on the parameter measured and control used. For example, the threshold of a parameter or biomarker may refer to the point at which a physiological or psychological effect begins to be produced or to be absent compared to a healthy control, and where the value is considered to be significantly different from the healthy control (i.e., the subject is considered to not be healthy, e.g., suffers from infertility). In the context of this application, the threshold or threshold value may be used to set a pre-determined threshold that can be used in the clinical setting to determine whether a patient suffers from infertility and is eligible to receive fertility treatment. In other words, this may be a value, or range of values, determined from analysis of control (healthy) individuals, which is used to benchmark clinical test subjects and to determine whether the test subject is healthy or has the condition, depending on whether their clinical readings fall within or outside of the control readings or ranges. Hence, the threshold value may be set by a respective control and correspond to a mean value determined within a control group.

As used herein, the term “fertility treatment” means any treatment or medical procedure intended to increase the likelihood of a person successfully conceiving a child. Within the context of this application and, in particular, male infertility, “fertility treatment” may refer to the treatment of low levels of testosterone (hypogonadism) wherein the treatment comprises administering to the patient, for example, anabolic-androgenic steroids, human chorionic gonadotropin (hCG), clomiphene citrate, aromatase inhibitors, and/or androgen receptor modulators.

As used herein, the term “infertility” refers to a disease of the male or female reproductive system defined by the failure to achieve a pregnancy after 12 months or more of regular unprotected sexual intercourse. Fertility care encompasses the prevention, diagnosis and treatment of infertility. In the male reproductive system, infertility may be caused by problems in the ejection of semen, absence or low levels of sperm, or abnormal shape (morphology) and movement (motility) of the sperm. Problems with male fertility can be caused by a number of health issues and medical treatments including, but not limited to, varicocele, infection, ejaculation issues, antibodies that attack sperms, tumours, undescended testicles, hormone imbalances, defects of tubules that transport sperm, chromosomal defects, coeliac disease, medications, and prior surgeries (e.g., vasectomy, inguinal hernia repairs, scrotal or testicular surgeries, prostate surgeries). Overexposure to certain environmental elements such as heat, toxins and chemicals can reduce sperm production or sperm function (e.g., industrial chemicals, heavy metal exposure, radiation, overheating of testicles). Health, lifestyle and other causes may also contribute to infertility or sub-fertility. As used herein the term “sub-optimal fertility” refers to medical condition wherein couples are not able to conceive a child even though they have had frequent, unprotected sexual intercourse for a year or longer. Systemic disease, external factors (e.g., drugs, lifestyle, etc.), or combinations of these also result in male subfertility (Hirsh, 2003).

The term “hypogonadotropic hypogonadism” or “HH” is characterized by failure of gonadal function secondary to deficient gonadotropin secretion, resulting from either a pituitary or hypothalamic defect. Although the genetic basis for idiopathic hypogonadotropic hypogonadism is largely unknown, mutations in several genes involved in the hypothalamo-pituitary-gonadal axis development and function have recently been implicated in the pathogenesis of this condition. Genes currently recognized to be involved include KAL-1 (associated with X-linked Kallmann Syndrome), gonadotropin-releasing hormone (GnRH) receptor, gonadotropins, pituitary transcription factors (HESX1, LHX3, and PROP-1), orphan nuclear receptors (DAX-1, associated with X-linked adrenal hypoplasia congenital, and SF-1), leptin, leptin receptor, and prohormone convertase 1 (PC1) (Silveira et al., 2002).

The term “ultrasound” or “US” refers to sound waves with frequencies higher than the upper audible limit of human hearing. It is generally understood that ultrasound devices operate with frequencies from 20 kHz up to several gigahertz (GHz). At atmospheric pressure, ultrasonic waves have wavelengths of 1.9 cm or less. The ultrasound data can be ultrasound radiofrequency (“RF”) data, ultrasound in-phase quadrature (“IQ”) data, or the like. In general, the ultrasound data contains one or more spatial dimensions, which may include a lateral dimension, an axial dimension, an elevational dimension, and combinations thereof. For instance, the ultrasound data can contain two spatial dimensions, such as the lateral and axial dimensions. The ultrasound data may also contain a temporal dimension, such as a dimension in slow time (i.e., the temporal direction along which multiple ultrasound signals are collected).

As used herein, the term “CEUS” refers to contrast-enhanced ultrasound. CEUS is the application of ultrasound contrast medium to traditional medical sonography. Ultrasound contrast agents rely on the different ways in which sound waves are reflected from interfaces between substances. This may be the surface of a small air bubble or a more complex structure.

As used herein, the term “microbubbles” refers to microbubble contrast agents for use in contrast-enhanced ultrasound (CEUS). Hence, in one embodiment, sound is emitted by microbubbles, after ultrasound excitation. Pulse inversion can be used to enhance the power of signals emitted by microbubbles compared to tissue. Microbubble contrast agents are physiologically inert, non-toxic and pass through the pulmonary circulation following intravenous injection. Microbubbles as contrast materials require only a small dosage and show excellent detection sensitivity. Targeting ligands on the surface of microbubbles may permit the selective accumulation of these particles in the areas of interest, which show an up-regulated level of receptor molecules on vascular endothelium. Microbubbles contain either air or an inert gas, encapsulated either by a thin shell composed of a biocompatible material such as a lipid, protein or more recently, synthetic polymer (Berry et al., 2015). Some commercially available microbubble ultrasound contrast agents may include, but are not limited to, Sonazoid™ (lipid-stabilised perfluorobutane), Optison™ (uses perfluoropropane gas), SonoVue™ (sulphur hexafluoride encased by a phospholipid), and Definity™ (lipid-stabilised octafluoropropane gas) (Berry et al., 2015). According to the invention, the microbubbles have a mean diameter of about 2.5 μm, with 90% having a diameter less than 6 μm and 99% having a diameter less than 11 μm.

As used herein, the term “SRUS” refers to super-resolution ultrasound imaging. SRUS enables in vivo microvascular imaging of deeper-lying tissues and organs below the diffraction limit of conventional ultrasound.

As used herein, the terms “prevent”, “preventing” and “prevention” in the context of the present invention and the administration of a therapy(ies) to a subject refers to the inhibition of the development or onset of a disease or a symptom thereof. In one embodiment, it relates to the administration of the compound to a patient who is known to have an increased risk of developing a certain condition, disorder, or disease.

As used herein, the terms “treat”, “treatment”, and “treating” refer in the context of the present invention to the administration of the compound to a patient, which has already developed signs and/or symptoms of a certain condition, disorder, or disease. Beneficial or desired clinical results include, but are not limited to, alleviation of symptoms; diminishment of the extent of a condition, disorder, or disease stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects.

The terms “subject” or “patient” are used interchangeable and relate to an animal (e.g., mammals) that may need administration of the compound of the invention in the field of human or veterinary medicine. In specific embodiments, the subject is a human.

As used herein, the term “pharmaceutically acceptable” means approved by a regulatory agency. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. The formulation should suit the mode of administration.

As used herein, the term “effective amount” in the context of administering a therapy to a subject refers to the amount of a therapy which has a prophylactic and/or therapeutic effect(s).

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used.

### Methods of Selecting and Diagnosing a Male Subject

The present invention relates to different methods used in the diagnosis and assessment of male infertility and in the identification and selection of those men that would benefit from fertility treatment. Generally, the present invention provides methods for assessing testicular function by measuring the density of blood microvessels within the testes using CEUS/SRUS and methods of determining a health condition by automated processing of CEUS/SRUS microvascular images. Based on the specific implementation of CEUS/SRUS in reproductive imaging, biomarker measurements taken from the testes can be used to distinguish control from test subjects in disorders of the reproductive tissue. For example, the inventors have discovered in a proof-of-concept study that ultrasound data acquired from the testes in male subjects who have been administered a microbubble contrast agent can be used to diagnose and assess hypogonadism to select suitable patients for fertility treatment. The inventors of the present application surprisingly found that CEUS/SRUS in reproductive imaging of microvessels provides an effective method for assessing male subjects for hypogonadism, diagnosing male subjects with infertility, and selecting them for appropriate fertility treatment. Specifically, the inventors submit that the particular combination of CEUS/SRUS-mediated imaging of microvessels and detection of biomarkers (e.g., microvessel density, tortuosity, and diameter) provides an improved method of assessing, diagnosing, and selecting male subjects in need of fertility treatment.

Accordingly, provided herein is a method of selecting a male subject for fertility treatment, wherein the subject has been examined using contrast-enhanced ultrasound (CEUS) and/or super-resolution ultrasound (SRUS, also known as Ultrasound Localisation Microscopy or ULM), and wherein following examination, one or more testicular parameters have been determined for the subject, wherein the method comprises the following steps in the following order:
(a) comparing the one or more parameters of the subject to one or more threshold values of the one or more parameters;
(b) determining a difference between the one or more parameters of the subject and the threshold values; and
(c) selecting the subject for treatment when the one or more of the parameters of the subject are below or above the threshold values.

Also provided herein is a method for diagnosing or aiding in the diagnosis of hypogonadism in a male subject, wherein, following examination using contrast-enhanced ultrasound (CEUS) and/or super-resolution ultrasound (SRUS), a test profile of one or more testicular parameters has been determined for the subject; and wherein the method comprises the following steps in the following order:
(a) comparing the test profile of the subject to one or more threshold values of the one or more parameters;
(b) determining a difference between the test values of the subject and the threshold values; and
(c) diagnosing hypogonadism based on the comparison of the test profile with the threshold values.

For men struggling to conceive with their partners, diagnostic tools are limited. Baseline test, such as the “gold-standard” assessment to diagnose male hypogonadism, serves as an estimation of male fertility, leaving patients and clinicians in need of additional diagnostic methods and biomarkers. For selecting and diagnosing male subjects, as well as assessing male infertility, the status of different parameters, i.e., biomarkers, can be determined alone or in combination.

As used herein, healthy control subjects comprise male subjects who do not have hypogonadism and/or do not suffer from infertility. That is, these subjects provide information on “normal” expression levels of different parameters or biomarkers. Accordingly, in one embodiment, the healthy control is a male subject that is fertile. In one embodiment, the healthy control is a male subject that does not have hypogonadism. For example, men with a mean tortuosity of more than 3.396 C/L are less likely to be healthy men, and more likely to be hypogonadal or infertile men. Men with a mean diameter less than 75.74 μm are less likely to be healthy men, and more likely to be hypogonadal or infertile men. Men with a mean density ratio less than 0.057 (no units) are less likely to be healthy men, and more likely to be hypogonadal or infertile men. The threshold suggesting a distinction between patients and healthy controls are listed in Table 1.

TABLE 1
Parameters, including their units,
and approximated threshold values.

| | | Level suggestive that patient |
|---|---|---|
| Parameter | Units | is less likely to be healthy |
| Mean tortuosity | Ratio; C/L | More than 3.396 |
| Mean diameter | μm | Less than 75.74 |
| Mean vessel | Ratio; no pixels of | Less than 0.057 |
| density | vessels/no pixels on | |
| | region of interest (ROI) | |

Microvessel density (MVD) is a promising prognostic factor in management of patients with prostate cancer (Dabbs. (2006). Chapter 14—Immunohistology of the Prostate, Bladder, Testis and Kidney. Diagnostic Immunohistochemistry (2^{nd} Edition)). The inventors of the instant application has discovered that microvessel density can be used as a prognostic indicator or biomarker for patients with hypogonadism. Notably, microvessel density, is measured as a relative number, which depends on contrast agent dose and acquisition time. According to the invention, microvessel density is measured and expressed as a ratio (no pixels of vessels/no pixels on region of interest (ROI)). Microvessel density can be determined independently for the right and left testes. In one embodiment, mean microvessel density is measured. In one embodiment, microvessel density of the left testis is measured. In one embodiment, microvessel density of the right testis is measured. In one embodiment, mean microvessel density of the right and/or left testes is measured in all test subjects.

In the following, the numbers and ranges mentioned for a mean microvessel density are also applicable to the microvessel density of a single value and vice versa.

Microvessel density may be expressed as a percentage of the detected vessel area over the full area. The microvessel density may be between 0% and 100%. In one embodiment, the microvessel density is about 90%, 80%, 70%, 60%, 50%, 40%, 30%, 20%, or 10%. In one embodiment, the microvessel density is less than 10%, 20%, 30%, 40%, 50%, 60%, 70%, 80%, or 90%.

In some embodiments, microvessel density in the test subject or subjects is measured relative to a healthy or normal subject or healthy or normal population. The microvessel density may be expressed as a relative different between test subject and healthy control. The microvessel density may be expressed or measured as a relative change in density between the test subject and healthy control.

In one embodiment, the microvessel density in the test subject is decreased compared to control. In one embodiment, the microvessel density in a test subject is decreased compared to a healthy control. In one embodiment, the microvessel density in the right and/or left testes is significantly decreased in a test subject compared to a healthy control. In one embodiment, microvessel density of the left and/or right testes or the mean thereof is decreased compared to a healthy control. In one embodiment, microvessel density of the left testis is decreased in a test subject compared to a healthy control. In one embodiment, microvessel density of the right testis is decreased in a test subject compared to a healthy control.

In one embodiment, the mean microvessel density of the left testis of all test subjects is decreased compared to healthy controls. In one embodiment, the mean microvessel density of the right testis of all test subjects is decreased compared to healthy controls.

In one embodiment, the mean microvessel density of the left and right testes of all test subjects is decreased compared to healthy controls. In one embodiment, the mean microvessel density of the right and left testes is significantly decreased in a test subject compared to healthy control.

In one embodiment, the mean microvessel density threshold value is from about 0.10 to about 0.15, such as 0.11 to 0.14. In one embodiment, the mean microvessel density threshold value is about 0.10, 0.11, 0.12, 0.13, 0.14, or 0.15. In one embodiment, the mean microvessel density threshold value is about 0.10. In one embodiment, the microvessel density threshold value is about 0.10.

In one embodiment, mean microvessel density of the right and left testes in a test subject is below about 0.1, 0.09, 0.08, 0.07, 0.06, 0.05. 0.04, 0.03, 0.02 or 0.01. In a preferred embodiment, mean microvessel density of the right and left testes in a test subject is about 0.04.

In one embodiment, mean microvessel density of the right and left testes of all test subjects is below about 0.1, 0.09, 0.08, 0.07, 0.06, 0.05. 0.04, 0.03, 0.02 or 0.01. In a preferred embodiment, mean microvessel density of the right and left testes of all test subjects is about 0.04.

In one embodiment, a microvessel density of below a threshold value of about 0.057 is indicative of hypogonadism. In one embodiment, a microvessel density below a threshold value when compared to control is indicative of hypogonadism. In one embodiment, a microvessel density below about 0.06, 0.05. 0.04, 0.03, 0.02 or 0.01 is indicative of hypogonadism. In one embodiment, a microvessel density in the left and/or right testes of below about 0.057 is indicative of hypogonadism.

In one embodiment, microvessel density in a test subject is between about 0 and 0.10, about 0 and 0.09, about 0 and 0.08, about 0 and 0.07. In one embodiment, microvessel density in a test subject is below about 0.1, 0.09, 0.08, 0.07, 0.06, 0.05. 0.04, 0.03, 0.02 or 0.01. In one embodiment, microvessel density in a test subject is below about 0.06, 0.05. 0.04, 0.03, 0.02 or 0.01. In one embodiment, the microvessel density in the test subject is less than about 0.057. In one embodiment, a microvessel density of 0.057 suggests that patient is less likely to be healthy.

Arterial tortuosity, that is, the presence of abnormal twists and turns of one or several arteries has been recognized for decades and associated with older age, female sex, high blood pressure, and other cardiovascular risk factors (Ciurica et al., 2019). More importantly, arterial tortuosity syndrome is a disorder that affects connective tissue, which provides strength and flexibility to structures throughout the body, including blood vessels. The inventors of the instant application submit that tortuosity is a prognostic indicator or biomarker for patients with hypogonadism. Microvessel tortuosity is expressed as the arc-chord ratio: the ratio of the length of the curve (C) to the distance between its ends (L, length), wherein arc-chord ratio equals 1 for a straight line and is infinite for a circle. The equation should be: t=C/L. Microvessel tortuosity can be determined independently for the right and left testes.

In the following, the numbers and ranges mentioned for a mean microvessel tortuosity are equally applicable to a single value of microvessel tortuosity and vice versa.

In one embodiment, the microvessel tortuosity of the left and/or right testes or the mean thereof is increased compared to the healthy control. In one embodiment, the microvessel tortuosity of the left testis or the mean thereof is increased compared to the healthy control. In one embodiment, the microvessel tortuosity of the right testis or the mean thereof is increased compared to the healthy control.

In one embodiment, a microvessel tortuosity above a threshold value when compared to control confirms a diagnosis of hypogonadism. In one embodiment, the microvessel tortuosity is increased in the test subject compared to a healthy control. In one embodiment, the microvessel tortuosity in the right and/or left testes is significantly increased in a test subject compared to a healthy control. In one embodiment, the mean microvessel tortuosity is significantly increased in a test subject compared to a healthy control.

In one embodiment, the mean microvessel tortuosity is about 3.0 C/L. In one embodiment, the microvessel tortuosity is between about 2.7 and 5.0 C/L for the right and/or left testes. In one embodiment, a mean microvessel tortuosity for the right and/or left testes of above about 3.4 C/L is indicative of hypogonadism. In one embodiment, a mean microvessel tortuosity for the right and/or left testes above about 3.4 C/L is indicative of hypogonadism. In one embodiment, a mean microvessel tortuosity for the right and/or left testes above about 3.4 C/L is indicative of hypogonadism. In one embodiment, a mean microvessel tortuosity for the right and/or left testes of above about 3.2 C/L is indicative of hypogonadism. In one embodiment, a mean microvessel tortuosity for the right and/or left testes above about 3.2 C/L is indicative of hypogonadism. In one embodiment, a mean microvessel tortuosity for the right and/or left testes above about 3.2 C/L is indicative of hypogonadism.

In one embodiment, the mean microvessel tortuosity of the right and/or left testes in a test subject is between about 2.7 and 5.0 C/L. In one embodiment, the mean microvessel tortuosity of the right and/or left testes in a test subject is between about 3.5 and 4.6 C/L. In one embodiment, the mean microvessel tortuosity of the right and/or left testes in a test subject is about 3.0 C/L, about 4.0 C/L or about 5 C/L. In one embodiment, the mean microvessel tortuosity of the right and/or left testes in a test subject is about 2.7 C/L, 2.8 C/L, 2.9 C/L, 3.0 C/L, 3.1 C/L, 3.2 C/L, 3.3 C/L, 3.4 C/L, 3.5 C/L, 3.6 C/L, 3.7 C/L, 3.8 C/L, 3.9 C/L, 4.0 C/L, 4.1 C/L, 4.2 C/L, 4.3 C/L, 4.5 C/L, 4.6 C/L, 4.7 C/L, 4.8 C/L, 4.9 C/L, or 5.0 C/L. In one embodiment, the mean microvessel tortuosity of a test subject is about 4.0 C/L.

In one embodiment, the mean microvessel tortuosity of the right and left testes of all test subjects is above about 3.0 C/L, about 3.1 C/L, about 3.2 C/L, about 3.3 C/L, about 3.4 C/L, about 3.5 C/L, about 3.6 C/L, about 3.7 C/L or about 3.8 C/L. In a preferred embodiment, the mean microvessel tortuosity of the right and left testes of all test subjects is about 3.0 C/L, about 3.1 C/L, about 3.2 C/L, about 3.3 C/L, about 3.4 C/L, about 3.5 C/L, about 3.6 C/L, about 3.7 C/L or about 3.8 C/L.

In one embodiment, the microvessel tortuosity of the right and/or left testes in a test subject is between about 3.5 and 4.6 C/L. In one embodiment, the tortuosity of the right and/or left testes in a test subject is about 4.0 C/L. In one embodiment, the tortuosity of the right and/or left testes in a test subject is about 3.5 C/L, 3.6 C/L, 3.7 C/L, 3.8 C/L, 3.9 C/L, 4.0 C/L, 4.1 C/L, 4.2 C/L, 4.3 C/L, 4.4 C/L, 4.5 C/L, or 4.6 C/L. In one embodiment, the tortuosity of a test subject is about 4.0 C/L.

In one embodiment, a microvessel tortuosity above a threshold value when compared to a healthy control confirms a diagnosis of hypogonadism. In one embodiment, a microvessel tortuosity above a threshold value of about 3.2 C/L in a test subject when compared to a healthy control confirms a diagnosis of hypogonadism. In one embodiment, a microvessel tortuosity of the left and/or right testes of above about 3.2 C/L is indicative of hypogonadism. In one embodiment, a microvessel tortuosity of the left and/or right testes of above about 3.3 C/L is indicative of hypogonadism. In one embodiment, a microvessel tortuosity of the left and/or right testes of above about 3.4 C/L is indicative of hypogonadism. In one embodiment, a microvessel tortuosity above a threshold value of about 3.5 C/L in a test subject when compared to a healthy control confirms a diagnosis of hypogonadism. In one embodiment, a microvessel tortuosity above a threshold value of about 3.6 C/L in a test subject when compared to a healthy control confirms a diagnosis of hypogonadism.

According to the invention, men with a microvessel tortuosity of more than 3.396 (Ratio; C/L) are less likely to be healthy men, and hence, are more likely to be hypogonadal or infertile men. In one embodiment, a tortuosity of more than 3.396 C/L suggests that patient is less likely to be healthy. In one embodiment, the tortuosity of the test subject in the right and/or left testes is more than 3.396 C/L. In one embodiment, a tortuosity of more than about 4.0 C/L suggests that patient is less likely to be healthy. In one embodiment, the tortuosity of the test subject in the right and/or left testes is more than about 4.0 C/L. In one embodiment, the tortuosity in the right and/or left testes is more than about 3.0 C/L, 3.1 C/L, 3.2 C/L, 3.3 C/L, 3.4 C/L, 3.5 C/L, 3.6 C/L, 3.7 C/L, 3.8 C/L, 3.9 C/L, 4 C/L, 4.1 C/L, 4.2 C/L, 4.3 C/L, 4.4 C/L, 4.5 C/L, 4.6 C/L, 4.7 C/L, 4.8 C/L, 4.9 C/L, or 5.0 C/L in a test subject. In one embodiment, the tortuosity in the right and/or left testes is 3.0 C/L, 3.1 C/L, 3.2 C/L, 3.3 C/L, 3.4 C/L, 3.5 C/L, 3.6 C/L, 3.7 C/L, 3.8 C/L, 3.9 C/L, 4 C/L, 4.1 C/L, 4.2 C/L, 4.3 C/L, 4.4 C/L, 4.5 C/L, 4.6 C/L, 4.7 C/L, 4.8 C/L, 4.9 C/L, or 5.0 C/L in a test subject. In one embodiment, the mean microvessel tortuosity threshold value is about 3.0 C/L. In one embodiment, the mean microvessel tortuosity threshold value is about 4.0 C/L.

Diameters of microvessels undergo continuous structural adaptation in response to hemodynamic and metabolic stimuli. It is known from the art that microcirculation has a significant role in disease development and progression, e.g., prostate cancer. The inventors of the application have further discovered that microvessel diameter can be used to identify those subjects that suffer from male infertility, i.e., hypogonadism. Microvessel diameter is measured in μm and can be determined independently for the right and left testes. In one embodiment, microvessel diameter in the left and/or right testes or the mean thereof is decreased in the test subject compared to the healthy control.

In the following, the numbers and ranges mentioned for a mean microvessel diameter are also applicable to the microvessel diameter of a single value and vice versa.

In one embodiment, microvessel diameter of the left testis or the mean thereof is decreased in the test subject compared to the healthy control. In one embodiment, microvessel diameter of the left testis or the mean thereof is decreased compared to the healthy control. In one embodiment, microvessel diameter of the right testis or the mean thereof is decreased in the test subject compared to the healthy control.

In one embodiment, a microvessel diameter below a threshold value when compared to healthy control confirms a diagnosis of hypogonadism. In one embodiment, the microvessel diameter is decreased in the test subject compared to healthy control. In one embodiment, the microvessel diameter in the left and/or right testes is significantly decreased in the test subject compared to healthy control. In one embodiment, the microvessel diameter in the left and/or right testes or the mean thereof is decreased compared to the healthy control. In one embodiment, the mean microvessel diameter is significantly decreased in the test subject compared to healthy control.

In one embodiment, a microvessel diameter below a (mean) threshold value when compared to healthy control confirms a diagnosis of hypogonadism. In one embodiment, the microvessel diameter threshold value is about 70-120 μm. In one embodiment, a mean microvessel diameter is below a threshold value of about 70 μm. In one embodiment, a mean microvessel diameter is below a threshold value of about 75 μm. In one embodiment, a mean microvessel diameter is below a threshold value of about 80 μm. In one embodiment, a mean microvessel diameter is below a threshold value of about 85 μm. In one embodiment, a mean microvessel diameter is below a threshold value of about 90 μm. In one embodiment, a mean microvessel diameter is below a threshold value of about 95 μm. In one embodiment, a mean microvessel diameter is below a threshold value of about 100 μm. In one embodiment, the mean microvessel diameter threshold value is about 76 μm. In one embodiment, the mean microvessel diameter threshold value is about 75 μm. In one embodiment, the mean microvessel diameter is about 50, 60, 70, 80, or 90 μm. In one embodiment, the mean microvessel diameter is below about 100, 90, 80, 70, 60, 50, or 40 μm. In one embodiment, the mean microvessel diameter does not exceed about 40, 50, 60, 70, 80, 90, or 100 μm. In one embodiment, the mean microvessel diameter does not exceed 55 μm. In one embodiment, the mean microvessel diameter threshold is at about 50, 51, 52, 53, 54, 55, 56, 57, 58, 59, or 60 μm. In one embodiment, the mean microvessel diameter threshold is at about 75.74 μm. In one embodiment, the microvessel diameter threshold is at about 75.74 μm.

In one embodiment, the mean microvessel diameter of the right and left testes decreased from about 90 μm in the control group to about 70 μm in the patient group. In one embodiment, the mean microvessel diameter of the right and left testes decreased to about 56 μm or below. In one embodiment, the microvessel diameter of the right and left testes decreased to about 75.74 μm or below.

In one embodiment, a microvessel diameter in the left and/or right testes below about 76 μm is indicative of hypogonadism. In one embodiment, a microvessel diameter in the left and/or right testes below about 75.74 μm, is indicative of hypogonadism. In one embodiment, a microvessel diameter in the left and/or right testes below about 75 μm, is indicative of hypogonadism. In one embodiment, microvessel diameter below about 70, 71, 72, 73, 74, 75, 76, 77, 78, 79, or 80 μm is indicative of hypogonadism. In one embodiment, a microvessel diameter is at about 75.74 μm. In one embodiment, a microvessel diameter of about 75.74 μm, suggests that patient is less likely to be healthy.

In one embodiment, the mean refers to the mean value of the right and left testes of a test subject. In one embodiment, the mean refers to the mean value of all test subjects with respect to the left testis. In one embodiment, the mean refers to the mean value of all test subjects with respect to the right testis. In one embodiment, the mean refers to the mean value of all test subjects with respect to the left and right testis.

In one embodiment, the mean refers to the mean value of the right and left testes of a healthy control. In one embodiment, the mean refers to the mean value of all healthy controls with respect to the left testis. In one embodiment, the mean refers to the mean value of all healthy controls with respect to the right testis. In one embodiment, the mean refers to the mean value of all healthy controls with respect to the left and right testis.

Furthermore, microvessel blood flow velocity may also be measured. In one embodiment, microvessel velocity is measured of the left and/or right testis. A microvessel velocity of less than 1.5 mm/sec or 1.4 mm/sec may suggest that patient is less likely to be healthy. In one embodiment, the test subject has a microvessel velocity of about 1.4 mm/sec, 1.3 mm/sec, 1.2 mm/sec, 1.1 mm/sec, or 1.0 mm/sec. In one embodiment, the healthy control has a microvessel velocity of more than about 1.5 mm/sec.

In one embodiment, the one or more parameters are selected from the list comprising testicular microvessel density, testicular microvessel diameter, testicular microvascular flow, regularity or entropy of testicular microvascular flow (e.g., flow velocity, flow direction and flow rate), testicular microvessel tortuosity, and spatial heterogeneity of these parameters. In one embodiment, the parameter is microvessel density (i.e., the number found per unit area of image). In one embodiment, the parameter is microvessel tortuosity. In one embodiment, the parameter is microvessel diameter. In one embodiment, the parameter is testicular microvascular flow. In one embodiment, the parameter is microvessel blood flow velocity, flow direction and/or flow rate. In one embodiment, the parameter is velocity of blood flow through microvessels. It is to be understood that any combination of said biomarkers can be determined. For example, any of the parameters mentioned above can be measured alone or in combination or in addition to testicular volume, which is currently the primary biomarker used to assess testicular function (by Prader orchidometry or by regular ultrasound). In one embodiment, one or more of these parameters are determined. In one embodiment, at least one of the parameters is determined. In one embodiment, one or more parameters is determined with one or more additional biomarkers. In one embodiment, imaging biomarkers are utilised alone or in combination to diagnose different disease indications, preferably male infertility.

In one embodiment, the treatment comprises administering to the male subject in need thereof an effective amount of an anabolic-androgenic steroid. In one embodiment, the anabolic-androgenic steroid is testosterone or a derivate thereof. In one embodiment, an effective amount of an anabolic-androgenic steroid may be administered in combination with another active ingredient.

Also provided herein is the use of compositions for the treatment of infertility. Specifically, provided herein is the use of compositions in the manufacture of a medicament for treating infertility or sub-optimal fertility of a male subject, wherein the composition comprises an anabolic-androgenic steroid, human chorionic gonadotropin (hCG), clomiphene citrate, aromatase inhibitors, or androgen receptor modulators, and wherein the subject has been determined to have reduced density, elevated tortuosity and/or reduced diameter of microvessels within the testes.

Methods of Treating Male Infertility and/or Sub-Fertility

The present invention also relates to methods of treating infertility. Specifically, provided herein are methods of treating infertility or sub-optimal fertility of a male subject. Provided herein is a method of treating infertility or sub-optimal fertility of a male subject, wherein the method comprises administering an effective amount to the subject in need thereof of an anabolic-androgenic steroid, human chorionic gonadotropin (hCG), clomiphene citrate, aromatase inhibitors, and/or androgen receptor modulators; and wherein the subject has been determined to have reduced density, elevated tortuosity and/or reduced diameter of microvessels within the testes. In one embodiment, treatment of infertility or sub-optimal fertility is associated with hypogonadism, cancer treatment, trauma, mumps infection, genetic disorder. In one embodiment, the method of treatment comprises testosterone therapy. In one embodiment, the method comprises enhancing testosterone activity in the adult male. In one embodiment, the method comprises administering to the male subject an effective amount of an anabolic-androgenic steroid or pharmaceutical composition comprising the same effective in reducing or eliminating hypogonadism in said subject. In one aspect, the method enhances testosterone activity in the male subject.

Provided herein are methods of treating infertility or sub-optimal fertility of a male subject, wherein the method comprises (i) examining the male subject using CEUS and/or SRUS, (ii) determining one or more testicular parameters, optionally wherein the parameter is microvessel density, tortuosity, and/or diameter, (iii) determining a difference between the one or more parameters and the one or more healthy controls; and (iv) administering to the male subject in need thereof an effective amount of an anabolic-androgenic steroid.

Provided herein are methods of treating infertility or sub-optimal fertility of a male subject, wherein the method comprises (i) injecting the male subject with a contrast agent as defined herein (ii) examining the male subject using CEUS and/or SRUS, (iii) determining one or more testicular parameters, (iv) determining a difference between the one or more parameters and the one or more healthy controls; and (iv) administering to the male subject in need thereof an effective amount of an anabolic-androgenic steroid if the one or more parameters significantly deviates from the health control. In one embodiment, the contrast agent is administered to the patient 30 minutes prior to examination. In one embodiment, the contrast agent is a microbubble contrast agent.

In a specific embodiment, pharmaceutical compositions are administered parenterally, orally, intradermally, or transdermally. In one embodiment, the pharmaceutical composition is administered using a mucoadhesive oral patch, transdermal film or gel. In one embodiment, testosterone is administered intramuscularly, transdermally, subcutaneously, nasally, buccally or orally.

The compositions described herein may be used in methods of treating infertility or sub-optimal fertility of a male subject.

### Methods for Assessing Testicular Function

The inventors of the present invention have developed a novel and inventive protocol for the assessment of testicular function using CEUS and/or SRUS.

Provided herein is a method for assessing testicular function in a male subject, the subject having been examined using contrast-enhanced ultrasound (CEUS) and/or super-resolution ultrasound (SRUS), and wherein following examination, one or more testicular parameters have been determined, and wherein the method comprises the following steps in the following order:
(a) comparing the one or more parameters to one or more threshold values of the one or more parameters;
(b) determining a difference between the one or more parameters and the one or more threshold values; and
(c) assessing testicular function based on the comparison of the one or more parameters with the one or more threshold values.

Further provided herein is a computer-implemented method for assessing testicular function, and or providing a diagnosis of hypogonadism, in a male subject based on processing of received CEUS and/or SRUS images. The method may comprise receiving one or more ultrasound image collected using contrast-enhanced ultrasound (CEUS) and/or super-resolution ultrasound (SRUS); determining one or more testicular parameters from the one or more ultrasound images; comparing the one or more parameters to one or more threshold values of the one or more parameters; determining a difference between the one or more parameters and the one or more threshold values; and outputting an indication of testicular function based on the comparison of the one or more parameters with the one or more threshold values.

Contrast agents and US imaging are increasingly used in combination for the assessment and tracking of biomarkers in vivo. In particular, the physical characteristics of microbubbles have allowed the development of a variety of contrast-specific imaging techniques. One simple method to distinguish bubbles from tissue is to excite the bubbles so as to produce harmonics and then detect these in preference to the fundamental frequency echo from tissue. Accordingly, in one embodiment, CEUS and/or SRUS use a microbubble contrast agent, and wherein the contrast agent was administered to the patient 30 minutes prior to examination. Hence, in one embodiment, the contrast agent is microbubbles. In some embodiments, different microbubbles (e.g., microbubbles with different sizes) with different resonant ultrasound frequencies can be used for imaging, so that by selecting a specific ultrasound frequency (e.g., either transmit or receive at a specific frequency), only a subgroup of selected microbubbles will be imaged, thereby forming ultrasound data containing isolated microbubble sources. The microbubbles may have a mean diameter of about 2.5 μm, with 90% having a diameter less than 6 μm and 99% having a diameter less than 11 μm. In one embodiment, the microbubbles have a mean diameter of about 2.5 μm. in one embodiment, the microbubbles have a mean diameter of about 2.5 μm with 90% having a diameter less than 6 μm and 99% having a diameter less than 11 μm. In one embodiment, the microbubbles have a mean diameter of about 2.5 μm with 99% having a diameter less than 11 μm.

The microbubble signal can be obtained from both the linear and nonlinear components of the ultrasound wave. The linear component is typically at the fundamental frequency of the applied US wave, while the nonlinear component can be at the harmonic frequencies of the applied US wave, at the fundamental frequency of the applied US wave, or both. For instance, the nonlinearity introduced by amplitude-modulation-based imaging methods can be at the fundamental frequency.

Ultrasound pulses can be measured at different frequencies. For example, US pulses can be measured at high frequency (40 MHz). At US frequencies in the 20-40 MHz range, it is possible to differentiate the blood in the microcirculation from the surrounding tissue (Cheung et al., 2008). Hence, according to the invention, the US measurements may be obtained at 15-20 MHz. In some embodiments, the measurements and objective microvascular flow are obtained at 18 MHz. Contrast Ultrasound images may be obtained at 6.0 MHz. Contrast Ultrasound images may be obtained at 7.0 MHz. Overall frequency range of the whole scan may range from 5-18 MHz. In some embodiments, the frequency range of the whole scan is 5-18 MHz. In some embodiments, the frequency is about 5-18 MHz. In some embodiments, US frequencies are measured between 2-40 MHz. In some embodiments, the frequencies are between 4-20 MHz. In some embodiments, the frequencies are between about 4-20 MHz.

US measurements can be taken in different orientations. In some embodiments, US scans are obtained in multiple orientations to ensure the imaging of the blood vessels. Testicular mapping can be based on different areas within the testis: lateral, medial, cranial, caudal, posterior and/or anterior.

Various contrast agents known to the skilled person may readily be applied within the context of the present invention. For example, commercially available microbubble ultrasound contrast agents may include, but are not limited to, Sonazoid™ (lipid-stabilised perfluorobutane), Optison™ (uses perfluoropropane gas), SonoVue™ (sulphur hexafluoride encased by a phospholipid), and Definity™ (lipid-stabilised octafluoropropane gas). Accordingly, in one embodiment, the contrast agent comprises lipid-stabilised perfluorobutane, perfluoropropane gas, sulphur hexafluoride, or octafluoropropane gas.

Sulphur hexafluoride (SonoVue®) is a gaseous contrast agent that is not soluble in body fluids or water. SonoVue® can be used for Doppler tests for large blood vessels, such as those in the head, those leading to the head or the main vein to the liver, or for smaller blood vessels such as those in areas of disease in the breast or liver. In one preferred embodiment, the contrast agent is sulphur hexafluoride.

Also provided herein are methods of determining one or more parameters associated with hypogonadism, wherein the method comprises (i) examining the left and/or right testes of a male subject using CEUS and/or SRUS using the techniques and protocols as set out herein, and (ii) determining and increase or decrease in one or more testicular parameters compared to a healthy control. In one embodiment, the one or more testicular parameters are microvessel density, microvessel tortuosity, and/or microvessel diameter.

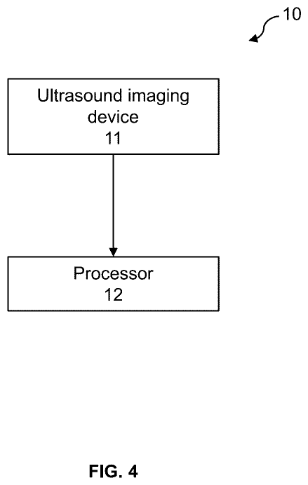

The methods of assessing testicular function and diagnosing a health condition, such as hypogonadism, described herein are preferably implemented by the automated analysis of images collect using contrast-enhanced ultrasound (CEUS) and/or super-resolution ultrasound (SRUS, also known as Ultrasound Localisation Microscopy or ULM). In particular, the ultrasound images may be processed using a computer-implemented method to determine one or more measurements of the microvessel morphology that are usable as biomarkers to assess testicular function. The image processing method may be fully or partially automated. In some examples the image processing method may be implemented by a system comprising the ultrasound imaging device. For example, FIG. 4 is a schematic illustration of a system 10 which includes an ultrasound imaging device 11 and a processor 12. The ultrasound imaging device 11 is arranged to collect ultrasound images, which may be by using contrast-enhanced ultrasound (CEUS) and/or super-resolution ultrasound (SRUS). The ultrasound images collected by the ultrasound imaging device 11 are received by the processor 12 to be processed according to the methods described herein. In other examples the image processing method may be carried out on a separate system, with the images received and processed according to them methods described herein. In some examples, the ultrasound imaging process itself may be partially or full automated. Possible implementation details for the software implemented methods for assessing testicular function and providing a diagnosis are set out below.

As described above, contrast-enhanced ultrasound (CEUS) and/or super-resolution ultrasound (SRUS) images of testicles are obtained, using the parameters set out herein. Following acquisition, the ultrasound images are then input to the software implemented method to extract the parameters usable as biomarkers and compare the parameters to determine a health condition indication. The software may be implemented in a computer connected to the ultrasound system, such that the collected images are processed locally to output the assessment. Alternatively, the image data may be sent to a separate computer running the analysis software.

As described above, the ultrasound image data preferably comprises a sequence of images of the same area of tissue, i.e. a video clip comprising a plurality of images in sequence. As known in the art, the structure of microvasculature cannot be resolved using conventional ultrasound imaging due to the fundamental diffraction limit at clinical ultrasound frequencies. It is possible to overcome this resolution limitation by localising individual microbubbles through multiple frames and forming a superresolved image. The ultrasound image data preferably comprises first image data visualising the maximum longitudinal plane for an imaging duration (for example 60 seconds), and second image data visualising the transverse plane of the testis for the same duration. The ultrasound image data preferably comprises microbubble contrast enhanced images, with a frequency range of 5-18 MHz. The image data is input into the software-implemented processing method, which may involve the following steps.

Initially, a region of interest withing the images may be determined. The region of interest may be selected by an operator. For example, a characteristic image may be displayed on a user interface and allow for selection of a region of interest to be input by a user. In other examples, the region of interest may be determined automatically. In particular, an area of the testis for analysis may be detected automatically, for example using feature recognition to centre the region of interest on an area comprising microvessel for analysis.

Preferably the method comprises a step of correcting for motion artefacts present in the sequence of images. Since the image data generally must be collected over seconds to minutes of acquisition in order to localise the microbubbles and resolve the microvascular image, motion is inevitable due to movement of the tissue and/or probe between images, and therefore preferably the image sequence is processed to remove or reduce motion artefacts. This preferably involves registration between images and applying a correction to estimate for and correct the movement of the tissue. The method may use, for example, a 2D phase correlation-based rigid geometric image registration method. The method may use, for example, the method described in S. Harput et al., “Two-Stage Motion Correction for Super-Resolution Ultrasound Imaging in Human Lower Limb,” in IEEE Transactions on Ultrasonics, Ferroelectrics, and Frequency Control, vol. 65, no. 5, pp. 803-814, May 2018.

The next step involves isolating the microbubble trajectories within the ultrasound images. In particular the method involves processing the images to isolate the microbubble signal from the background. Preferably the microbubble signal isolated using normalised cross-correlation. The method involves identifying a blinking microbubble signal caused by microbubble movement, disruption and dissolution. This microbubble signal may be extracted by a subtraction of immediately adjacent images (i.e. frame-to-frame subtraction) within the image data (in phase quadrature (IQ) data), to remove the background tissue signal and constant microbubble signals. One possible method for analysing the image data to isolate the microbubble signals is described in Song P et a. Improved Super-Resolution Ultrasound Microvessel Imaging With Spatiotemporal Nonlocal Means Filtering and Bipartite Graph-Based Microbubble Tracking. IEEE Trans Ultrason Ferroelectr Freq Control. 2018 February; 65(2):149-167.

The microbubble signal may then be processed to localise individual microbubbles, for example as described in the above referenced paper (Song P et al). This may involve thresholding and analysing the microbubble signal dimension and shape to determine the centre locations of microbubbles. A tracking algorithm may then be applied to track the trajectories of the microbubbles (for example as described in (Yan, T. Zhang, J. Broughton-Venner, P. Huang and M.-X. Tang, “Super-Resolution Ultrasound Through Sparsity-Based Deconvolution and Multi-Feature Tracking,” in IEEE Transactions on Medical Imaging, vol. 41, no. 8, pp. 1938-1947, August 2022).

A microvessel image may then preferably be generated based on the determined microbubbles tracks. In particular, the microbubble tracks may be used to generate a microvessel map, as described in the above references. The microvessel images may then be used to determine one or more parameters of the microvessel. In particular one or more measurements of the morphology of the vessels may be made. The inventors have determined, in particular that microvessel morphological parameters, particularly the vessel density, mean diameter and mean tortuosity, may be used as biomarkers for testicular function and diagnosis of hypogonadism. Microvessel density may be determined in an automated manner by calculating the ratio of the number of pixels comprising vessels to the total number of pixels in region of interest (ROI).

There can be challenges in accurately extracting the necessary morphological measurements. In particular, errors can be introduced when attempting to segment single vessels from the image to determine measurements. To reduce errors associated with segmentation, the vessel diameter may be determined by firstly determining a vessel centreline and subsequently, for each of a plurality of points along the centreline determining the diameter by doubling the distance to the nearest vessel wall. This process may be applied across the vessels of the image with a single vessel measured a plurality of times, with the possibility of a changing diameter along the flow direction. A mean vessel diameter may be calculated based on a plurality of such measurements, rather than attempting to segment and determine a single diameter for each vessel, avoiding the influence of error associated with segmenting single vessels from the image and branches from one vessel. In one example, the diameter may be calculated for each pixel on the centreline.

Similarly when measuring tortuosity the method may comprise measuring the tortuosity for a plurality of individual microbubble trajectories, rather than attempting to segment individual vessels and measuring the tortuosity. The tortuosity, being defined as the ratio of the length of the curve to the distance between its two ends, requires segmentation of the vessels and the two ends of a vessel to be determined. The difficulty in defining the ends of the vessels and branches can introduce errors and so, preferably the tortuosity measurements may be determined directly from the microbubbles trajectories, rather than from a microvessel image generated based on the microbubble signal.

The morphological measurements of the microvessel may be extracted automatically from the images and used as biomarkers for assessment of testicular function and diagnosis of hypogonadism. A key advantage of the present method is the suitability for automation to provide the automated analysis of the physiological data contained in the SRUS/CEUS images for providing a health condition prediction or diagnosis. It is possible to implement each of the above-describes image processing steps to provide an automated software-implemented method that takes as input a sequence of ultrasound images, processes the image to determine one or more testicular parameters and outputs an indication of predicted testicular function based on the comparison of the determined parameters to one or more threshold values. The above described testicular parameters are predicting of a number of conditions associated with testicular function. In particular the morphological parameters may be used to output an indication of hypogonadism and/or male infertility.

### Composition

The present invention also relates to compositions that can be used in the treatment of male infertility. The present invention provides composition for use in the treatment of hypogonadism in the adult male. In a specific embodiment, the compositions are pharmaceutical compositions. The pharmaceutical compositions described herein can be in any form that allows for the composition to be administered to a subject.

Depending on the results of the scan, different active compounds may be used. In some cases, treatment does not need to begin with testosterone or a derivate thereof. In some cases, treatment starts with testosterone or a derivate thereof.

Provided herein is a composition for use in treating infertility or sub-optimal fertility of a male subject, wherein the composition comprises an anabolic-androgenic steroid, human chorionic gonadotropin (hCG), clomiphene citrate, aromatase inhibitors, androgen receptor modulators; and wherein the subject has been determined to have reduced density, elevated tortuosity and/or reduced diameter of microvessels within the testes compared to healthy control. In one embodiment, the subject has been selected according to the method of the invention. In one embodiment, the subject has been diagnosed according to the method of the invention. In one embodiment, the subject has been assessed according to the method of the invention.

In one embodiment, the anabolic-androgenic steroid comprises testosterone or a derivative thereof. In one embodiment, treatment comprises enhancing testosterone activity in the adult male. In one embodiment, the composition is administered to the male subject in an effective amount. In one embodiment, the composition reduces or eliminates hypogonadism. In one embodiment, the composition enhances testosterone activity in the male subject.

As used herein, pharmaceutical composition means a mixture of substances suitable for administering to a subject. The pharmaceutical compositions described herein can be in any form that allows for the composition to be administered to a subject. In one embodiment, the subject is a male human. The compositions may be used in methods of treating male infertility or sub-optimal fertility of a male subject. In a specific embodiment, the pharmaceutical compositions are suitable for human administration. In some embodiments, a pharmaceutical composition may comprise one or more other therapies in addition to a composition of the invention. In one embodiment, a composition comprises a pharmaceutically acceptable carrier. In some embodiments, the pharmaceutically acceptable carrier can simply be a saline solution. This can be isotonic or hypotonic.

In one embodiment, the infertility or sub-optimal fertility is associated with hypogonadism, cancer treatment, trauma, mumps infection, and/or a genetic disorder. In one embodiment, infertility or sub-optimal fertility is associated with hypogonadism. In one embodiment, infertility or sub-optimal fertility is associated with cancer treatment. In one embodiment, infertility or sub-optimal fertility is associated with trauma. In one embodiment, infertility or sub-optimal fertility is associated with mumps infection. In one embodiment, infertility or sub-optimal fertility is associated with a genetic disorder.

In one embodiment, the anabolic-androgenic steroid is testosterone or a derivative thereof. Testosterone derivatives have been developed to enhance intrinsic androgenic potency, prolong duration of action, and/or improve oral bioavailability of synthetic androgens. Accordingly, in one embodiment, the derivative is testosterone ester. In one embodiment, the derivative is testosterone undecanoate. In one embodiment, the derivative is a dihydrotestosterone gel. In one embodiment, the derivative is testosterone undecanoate. In one embodiment, the derivate is testosterone enanthate and cypionate. In one embodiment, the derivative is testosterone propionate.

The amount of a composition which will be effective in the treatment of male infertility or sub-optimal fertility of a male subject will depend on the nature of the disease and can be determined by standard clinical techniques. One skilled in the art will appreciate that the constituents of the compositions for use may be varied in amounts. The doses administered are dependent on the route of administration and type of testosterone or derivate thereof given and should be decided according to the judgment of the practitioner and each subject's circumstances. In certain embodiments, an in vitro assay is employed to help identify optimal dosage ranges.

In one embodiment, the testosterone or derivative thereof is administered at a dose of 10-1000 mg/ml. In one embodiment, the testosterone or derivative thereof is administered by injection at a dose of 750 mg. In one embodiment, testosterone undecanoate is administered by injection at a dose of 750 mg followed by 750 mg after 4 weeks, followed by 750 mg every 10 weeks thereafter. In one embodiment, testosterone enanthate and cypionate are administered at a dose of 50 to 400 mg by injection every 2 to 4 weeks. In one embodiment, 2 to 6 pellets (75 mg each) of testosterone propionate implanted subcutaneously every 3 to 6 months. In one embodiment, a 30 mg patch is applied to the gum region twice a day. In one embodiment, a 40 mg transdermal gel is applied to the skin region daily.

Drugs may be administered to stimulate the body to make its own testosterone thus increasing the level of testosterone. Hence, in some embodiments, drugs are administered to stimulate the body to make its own testosterone. Treatment options may include selective oestrogen receptor modulators such as, for example, clomiphene citrate tablets. Hence, in one embodiment, treatment includes oestrogen receptor modulators. In one embodiment, treatment includes clomiphene citrate tablets. The usual dose range of clomiphene citrate tablets is 12.5 to 100 mg daily. In one embodiment, the dose range of clomiphene citrate tablets is 12.5 to 100 mg daily. Also, aromatase inhibitors such as anatrazole may be administered to increase testosterone levels. In one embodiment, anatrazole is administered. In some embodiments, the anatrazole dose range is 1 mg daily. In other instances, gonadotrophin hormones such as, for example, human chorionic gonadotrophin may be administered. In some embodiments, the doses range from 100-10000 IU per week in 2-4 divided doses.

Conventional pharmaceutically acceptable vehicles and techniques are used in preparing these dosage forms. In a specific embodiment, pharmaceutical compositions are formulated to be suitable for the intended route of administration to a subject. For example, the pharmaceutical composition may be formulated to be suitable for parenteral, oral, intradermal, or transdermal administration. In one embodiment, the testosterone is administered intramuscularly, transdermally, subcutaneously, nasally, buccally or orally. In one embodiment, the pharmaceutical composition is administered using a mucoadhesive oral patch, transdermal film or gel.

In one embodiment, the testosterone or derivative thereof is administered multiple times daily, daily to every 1 to 2 weeks, 3 to 4 weeks, every 6 to 12 weeks or every 3 to 6 months. In one embodiment, the testosterone or derivative thereof is administered every 2 to 4 weeks.

The pharmaceutical compositions described herein can be included in a container, pack, or dispenser together with instructions for administration for reducing or treating hypogonadism in an adult male suffering therefrom.

The aim of testosterone therapy is to achieve serum testosterone levels within the normal physiological range with dose adjustment to have the maximum effect on alleviation of symptoms. According, also provided here are methods of treating hypogonadism in a male subject.

### Patient Population

Male hypogonadism is increasing in prevalence, particularly in the older male population. Accordingly, in one embodiment, the subject is a human male subject. In one embodiment, a subject to be administered the composition described herein is a human male adult. In certain embodiments, a subject to be administered a composition described herein is an elderly human male. In certain embodiments, the subject is in need of treatment.

The inventors have surprisingly found that it is possible to select subjects with hypogonadism and assess hypogonadism using the methods of the invention. Moreover, the inventors have found that the methods of the invention may be used in diagnosing subjects with hypogonadism. In certain embodiments, the subject has been selected for fertility treatment using the methods according to the invention. In certain embodiments, testicular function in a male subject has been assessed using the methods of the invention. In certain embodiments, the subject has been diagnosed with hypogonadism using the methods of the invention.

In certain embodiments, a composition described herein is administered to a subject who has been selected for fertility treatment according to the methods of the invention. In certain embodiments, a composition described herein is administered to a subject who has been diagnosed with hypogonadism according to the methods of the invention. In certain embodiments, the subject has not been diagnosed with hypogonadism. In certain embodiments, a composition described herein is administered to a subject who was assessed for testicular function according to the methods of the invention.

In certain embodiments, the patient has been diagnosed with hypogonadism. In certain embodiments, an active compound or composition described herein is administered to a patient who has been diagnosed with hypogonadism. In certain embodiments, the subject has not been diagnosed with hypogonadism.

For example, aside from hypogonadism, male subject may experience other medical disorders, diseases, or conditions caused by, for example, cancer treatment, trauma, viral infections (e.g., mumps) and/or genetic disorders.

The present invention shall be described in more detail by the following Figures and Examples. The practice of the present invention will employ, unless otherwise indicated, conventional techniques of pharmacology and pharmaceutics, which are within the skill of the art.

### EXAMPLES

The examples shown in the following are merely illustrative and shall describe the present invention in a further way. These examples shall not be construed to limit the present invention thereto.

### Example 1. Detection of the Function of a Reproductive Organ Using CEUS/SRUS

A proof-of-concept trial (Phase 2b Clinical Trial) was set up to determine testicular function.

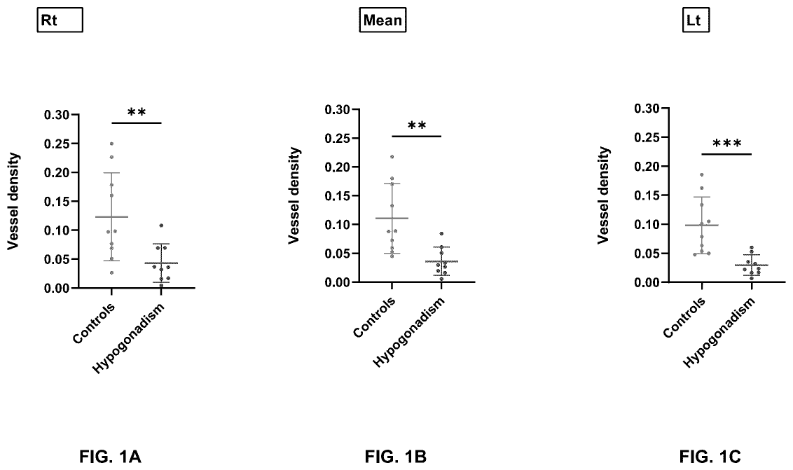

Twelve men with hypogonadotropic hypogonadism (HH; lack of hormonal stimulation of the testes causing low testosterone and low sperm count/infertility; also see, trial inclusion and exclusion criteria) and twelve healthy controls underwent CEUS/SRUS as specified below, assessing microvessel density, microvessel tortuosity and microvessel diameter of the left and right testes. In addition, mean values between right and left testes was determined, i.e., mean microvessel density, mean microvessel tortuosity and mean microvessel diameter. Healthy males were used as control, i.e., male subjects who do not have hypogonadism and/or do not suffer from infertility. The results of Example 1 are shown in FIGS. 1A to 3C.

Trial inclusion criteria: Male subjects with hypogonadotropic hypogonadism (HH), experiencing lack of hormonal stimulation of the testes causing low testosterone and low sperm count. Specifically, men aged 18-60 with low serum testosterone (<8 nmol/L) and a diagnosis of hypogonadotropic hypogonadism were included as test subjects (patients). These male subjects are considered infertile or sub-fertile. Men aged 18-60 with a normal sex hormone profile, semen analysis+/−previous fathered children are considered healthy volunteers.

Trial exclusion criteria: 1. Systemic comorbidities likely to affect results of study, e.g., established cardiovascular diseases, Insulin dependent Diabetes Mellitus with complications, Cushing's syndrome; 2. Surgical or structural abnormality on the testes likely to affect the results of the study; 3. Acute illness likely to affect the results of study; 4. Contraindications to microbubble agent, such as known hypersensitivity to the agent, history of Right-to-Left cardiac shunts, severe pulmonary hypertension (pulmonary artery pressure>90 mmHg), uncontrolled systemic hypertension; 5. Significant smoking history (>10 pack-years); 6. Impaired ability to provide full consent to take part in the study.

Contrast agent and administration: A transpulmonary echocardiographic contrast agent (SonoVue®) was administered through a vein in the antecubital fossa with the patient lying supine. Specifically, 8 microlitres/mL powdered sulphur hexafluoride contrast agent were used. Each mL of the dispersion contains 8 μL sulphur hexafluoride microbubbles, equivalent to 45 micrograms. The contrast agent was administered immediately after drawing into the syringe by injection into a peripheral vein. A volume of 2.4 ml was used to visualise each testis, which was given as a slow bolus over a period of 20-30 seconds. Every injection was followed by a flush with 5 mL of sodium chloride 9 mg/mL (0.9%) solution for injection. At this stage, patients were advised to try keep any movement to the minimum. During a single examination, a second injection of the recommended dose can be made when deemed necessary by the physician. The dose recommendations for intravenous administration also apply to elderly patients.

CEUS/SRUS: Following the administration of the contrast and at the point when the first MBs were seen on screen, the radiologist started recording a video clip visualising the maximum longitudinal plane for 60 seconds. Following this, another clip of same duration was recorded visualising the transverse plane of the testis. Thereafter, the exact same process was repeated for the contralateral testis. Contrast enhanced images were obtained using the clinical scanner Canon Aplio i800. All volume measurements and objective microvascular flow measurements are obtained at 18 MHz. Contrast Ultrasound images obtained at 6.0 MHz. Overall frequency range of the whole scan is from 5-18 MHz.

Statistical Analysis: Quantitative data were assessed for normality using the Shapiro-Wilk Normality test. Data were expressed as means, if normally distributed, and medians, if non-normally distributed. Statistical comparison was calculated between the means as independent samples T test for normally distributed data. For non-normally distributed data, statistical analysis was conducted using a Mann Whitney U Test. *≤0.05; **≤0.005; ***≤0.0005.

The different parameters determined and their units are listed in Table 2.

TABLE 2
Parameters measured and their respective units and abbreviations.

| Parameter | Units |
|---|---|
| Mean tortuosity | Ratio; (C/L) |
| Mean velocity | millimetre/second (mm/sec) |
| Mean diameter | micrometre (μm) |
| Mean vessel | Ratio; no pixels of vessels/no pixels |
| density | on region of interest (ROI) |

The microvascular morphology measurements were determined from the microvessel images. As shown in FIGS. 1A-1C compared to control (p<0.05). Mean microvessel density of the right testis and mean microvessel density of the left testis was significant decreased to below about 0.05 (see, FIGS. 1A and 1C). This decrease was significant when compared to healthy controls, which showed a mean vessel density of about 0.10 (see, FIGS. 1A and 1C; controls). Similarly, mean microvessel density of the right and left testes was reduced in patients suffering from Hypogonadism to about 0.05 when compared to control (FIG. 1B).

Furthermore, the inventors of the present application went on to determine microvessel tortuosity. Tortuosity is expressed as the arc-chord ratio: the ratio of the length of the curve (C) to the distance between its ends (L, length), wherein arc-chord ratio equals 1 for a straight line and is infinite for a circle:

$$t = C / L$$

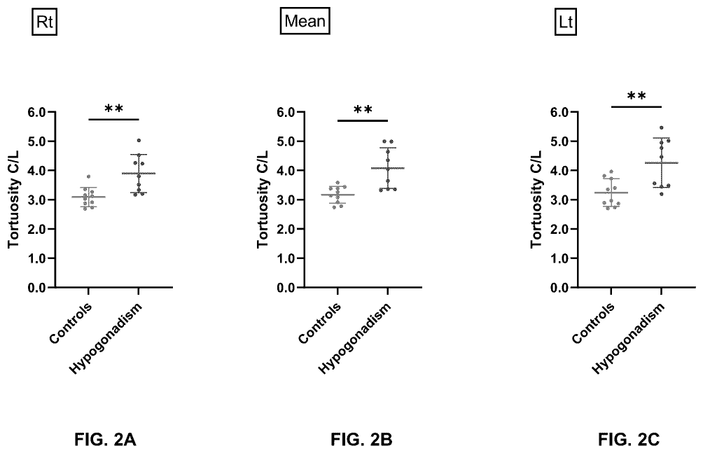

Assessing microvessel tortuosity, the inventors of the present application were able to show that patients suffering from hypogonadism have an increase in tortuosity when compared to controls (FIGS. 2A-2C). This increase was observed equally in the right and left testis (see, FIGS. 2A and 2C). Similarly, mean tortuosity of the right and left testes was increased from about 3 C/L in the control group to about 4 C/L in the patient group.

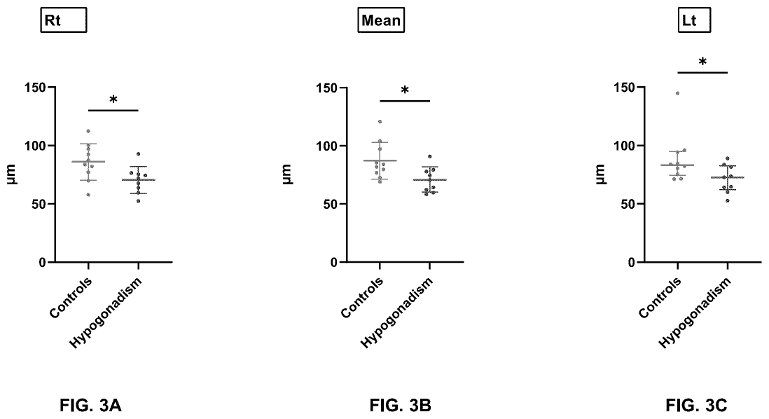

Assessment of (FIGS. 3A-3C) showed a general increase in microvessel diameter. The inventors were able to show a significantly decreased microvessel density in the right and left testis in patients suffering from hypogonadism (see, FIGS. 3A and 3C). The mean microvessel diameter of the right and left testes decreased from about 80 μm in the control group to about 60 μm in the patient group (FIG. 3B). The results show that CEUS/CRUS on testicular microvessels can be used to determine combinations of particular biomarkers and that there was a significant difference between the biomarkers in test subjects when compared to healthy control.

In summary, the inventors have shown that CEUS/SRUS can be used to determine one or more specific biomarkers (e.g., microvessel density, microvessel tortuosity, and microvessel diameter) in the testes to effectively identify and differentiate subjects suffering from male hypogonadism from healthy controls. Specifically, the inventors have successfully shown that these biomarkers can be used to reliably establish a disease status profile of male subjects and to select these subjects for subsequent fertility treatment. Moreover, the inventors have developed a novel and inventive approach of selecting male subjects for fertility treatment by examining the subject's testes using CEUS and/or SRUS. Additionally, the inventors have not only shown that the combination of CEUS/CRUS on testicular microvessels and particular biomarkers can be used for efficient assessment of testicular function in male subjects but that it can also be used in the diagnosis of male infertility, in particular male hypogonadism.

Those having ordinary skill in the art will appreciate that the disclosure can be modified in ways not specifically described herein.

1. A computer-implemented method for assessing testicular function in a male subject, the method comprising:
receiving one or more ultrasound images collected using contrast-enhanced ultrasound (CEUS) and/or super-resolution ultrasound (SRUS);
determining one or more testicular parameters from the one or more ultrasound images;
comparing the one or more parameters to one or more threshold values of the one or more parameters;
determining a difference between the one or more parameters and the one or more threshold values; and
outputting an indication of testicular function based on the comparison of the one or more parameters with the one or more threshold values.

2. The computer-implemented method of claim 1, further comprising:
determining a region of interest within the one or more ultrasound images and performing image-processing within the region of interest to determine the one or more testicular parameters.

3. The computer-implemented method of claim 2, further comprising:
outputting a diagnosis of hypogonadism based on the comparison of the one or more parameters with the one or more threshold values.

4. The computer-implemented method of claim 1, wherein the one or more testicular parameters each comprise a measurement of microvessel morphology based on the one or more ultrasound images.

5. The computer-implemented method of claim 4, wherein the method further comprises:
receiving a sequence of microbubble ultrasound images;
processing the sequence of ultrasound images to localise individual microbubbles through a sequence multiple images and thereby determine a plurality of microbubble tracks; and
determining the measurement of microvessel morphology based on the determined microbubble tracks.

6. The computer-implemented method of claim 5, wherein the method further comprises:
processing the sequence of ultrasound images to correct for motion in the image by performing image registration across the sequence of images; and
generating a microvascular image based on the motion-corrected sequence of ultrasound images.

7. The computer-implemented method of claim 1, wherein the one or more testicular parameters are selected from the list comprising:
testicular microvessel density, testicular microvessel diameter, testicular microvascular flow, regularity or entropy of testicular microvascular flow, and/or testicular microvessel tortuosity, and spatial heterogeneity of these parameters.

8. The computer-implemented method of claim 1, wherein the method further comprises:
processing the one or more ultrasound images to determine a microvessel density of the left and/or right testes or the mean thereof;
comparing the determined microvessel density to a threshold representing a healthy control; and
outputting a diagnosis of hypogonadism when the determined microvessel density is reduced compared to the threshold representing a health control.

9. The computer-implemented method of claim 8, wherein the method further comprises:
processing the one or more ultrasound images to determine a mean microvessel density;
comparing the determined mean microvessel density to a microvessel density threshold representing a healthy control, wherein the threshold is between 0.05 and 0.15; and
outputting a diagnosis of hypogonadism when the determined microvessel density is below the microvessel density threshold.

10. The computer-implemented method of claim 9, wherein the mean microvessel density threshold value is 0.057.

11. The computer-implemented method of claim 1, wherein the method further comprises:
processing the one or more ultrasound images to determine a microvessel tortuosity of the left and/or right testes or the mean thereof;
comparing the determined microvessel tortuosity to a microvessel tortuosity threshold representing a healthy control; and
outputting a diagnosis of hypogonadism when the determined microvessel tortuosity is greater than the threshold.

12. The computer-implemented method of claim 11, wherein the microvessel tortuosity threshold is 3.2 C/L.

13. The computer-implemented method of claim 12, wherein the method further comprises:
receiving a sequence of microbubble-contrast ultrasound images;
processing the sequence of microbubble-contrast images to determine a plurality of microbubble trajectories; and
calculating the tortuosity of the microbubble trajectories to determine the microvessel tortuosity.

14. The computer-implemented method of claim 1, wherein the method further comprises:
processing the one or more ultrasound images to determine a microvessel diameter in the left and/or right testes or the mean thereof;
comparing the determined microvessel diameter to a microvessel diameter threshold representing a healthy control; and
outputting a diagnosis of hypogonadism when the determined microvessel diameter is reduced compared to the threshold.

15. The computer-implemented method of claim 14, wherein the method further comprises:
processing the one or more ultrasound images to determine a microvessel diameter;
comparing the mean microvessel diameter to a mean microvessel diameter threshold, wherein the mean microvessel diameter is between 70 and 120 μm; and
outputting a diagnosis of hypogonadism when the determined mean microvessel diameter is below the mean microvessel diameter threshold.

16. The computer-implemented method of claim 1, wherein the method further comprises:
processing the one or more ultrasound images to generate a microvessel image; and
determining the one or more testicular parameters based on the microvessel image.

17. The computer-implemented method of claim 16, wherein the method further comprises:
receiving a sequence of ultrasound images collected using contrast-enhanced ultrasound (CEUS) and/or super-resolution ultrasound (SRUS) receiving a sequence of ultrasound images;
processing the sequence of ultrasound images to determine a microbubble signal;
generating the microvessel image using the determined microbubble signal; and
calculating the one or more testicular parameters using the microvessel images, wherein the one or more testicular parameters each comprise a measurement of a feature of a microvessel morphology.

18. (canceled)

19. A system comprising an ultrasound imaging device and a processor, wherein the processor is configured to:
receive one or more ultrasound images collected by the ultrasound imaging device using contrast-enhanced ultrasound (CEUS) and/or super-resolution ultrasound (SRUS);
determine one or more testicular parameters from the one or more ultrasound images;
compare the one or more parameters to one or more threshold values of the one or more parameters;
determine a difference between the one or more parameters and the one or more threshold values; and
output an indication of testicular function based on the comparison of the one or more parameters with the one or more threshold values.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A composition for use in treating infertility or sub-optimal fertility of a male subject, wherein the composition comprises an anabolic-androgenic steroid, human chorionic gonadotropin (hCG), clomiphene citrate, aromatase inhibitors, or androgen receptor modulators; and wherein the subject has been determined to have reduced density, elevated tortuosity and/or reduced diameter of microvessels within the testes compared to healthy control.

35. (canceled)

36. (canceled)

37. (canceled)

38. (canceled)

39. (canceled)

40. (canceled)

41. (canceled)

42. The system of claim 19, wherein the ultrasound imaging device is configured to obtain contrast-enhanced ultrasound (CEUS) and/or super-resolution ultrasound (SRUS) at a frequency range of 5-18 MHz.
